(19) United States
(12) Patent Application Publication
(10) Pub. No.: US 2016/0158505 A1
(43) Pub. Date: Jun. 9, 2016

(54) APPLICATOR FOR ANTISEPTIC OR MEDICATIONS

(71) Applicant: IP MED, INC., Oceanside, NY (US)

(72) Inventors: Jacob Jeffrey RAUCHWERGER, Lawrence, NY (US); Ari ISAACS, Oceanside, NY (US)

(21) Appl. No.: 14/903,912

(22) PCT Filed: Mar. 17, 2015
(86) PCT No.: PCT/US15/21036

§ 371 Date: Jan. 8, 2016

Related U.S. Application Data
(60) Provisional application No. 61/954,122, filed on Mar. 17, 2014.

(51) Int. Cl. A61M 25/09; A61M 29/02; A61L 2/00

(52) U.S. Cl. CPC A61M 25/09041; A61L 2/0082; A61M 29/02; A61M 2025/09116; A61M 2025/09133

(57) ABSTRACT

Disclosed herein is an applicator that can be advanced over an in-situ guidewire and into the subcutaneous tissue or tract prior to CVC placement or replacement, allowing sterilization of the subcutaneous tissue or tract before CVC introduction. The unique shape of the applicator allows sterilization of the skin surface as well as the subcutaneous tissue or tract of the original CVC that lies beneath the skin, which are difficult to reach and effectively sterilize with other applicators.

### BACKGROUND OF THE INVENTION

Central line associated blood stream infection (CLABSI) is a potentially devastating consequence of indwelling central venous catheters. It is estimated that a single CLABSI costs $35,000 to treat and associated complications (sepsis, endocarditis) can be life threatening. Since 2011, hospitals participating in the Center for Disease Control's National Healthcare Safety Network have been required to report their incidence of CLABSI. While the rates of CLABSI have declined in recent years with heightened awareness, chlorhexidine skin antiseptic, and antibiotic impregnated catheter use, it remains a vexing problem.

It had been known for decades that normal skin bacteria inhabit both the skin surface (also known as the stratum corneum) as well as structures that lie in deeper layers of the skin—including hair follicles, sweat glands and ducts and sebaceous glands and ducts. These bacteria are not eradicated by standard antimicrobial skin cleansing and preparation, as these antimicrobials do not reliably penetrate beyond the most superficial skin layer. Recent evidence in the medical literature suggests that these deep bacterial reservoirs often contaminate central venous catheters (CVCs) and may predispose to CLABSI development. Thus a need exists for a device that can effectively and reliably eradicate bacterial reservoirs in the deep layers of the skin prior to CVC placement.

In addition, patients with CVCs often require the CVCs to be changed during their hospital stay. This can be accomplished in one of two ways:

1) Placement of a new CVC at a new location (commonly called the ‘fresh stick’ technique); or

2) Removing the existing CVC over a guidewire and then using the guidewire to direct a new CVC through the skin and into the original CVC site Cover wire' technique).

Each technique has its advantages and disadvantages. The fresh stick technique carries a significantly lower rate of infection, but a higher rate of mechanical complications as a new central vein must be accessed for placement. On the other hand, the change over wire technique is technically much simpler and carries a lower mechanical complication rate but is associated with a much higher rate of CVC bacterial colonization and CLABSI.

Despite the fact that practitioners employ sterile technique during both de novo CVC placement as well as over the wire exchange, CLABSI occurs at a much higher rate with the over the wire technique than during de novo CVC placement. It is believed that bacteria colonize the path that the indwelling CVC follows from the skin to the central vein. This subcutaneous tract bacterial colonization (TBC) likely leads to contamination of the new CVC during over the line CVC exchange, accounting for the higher infection rates observed with this technique. Thus, a need clearly exists for a device capable of reducing infections during CVC over the wire exchanges.

### SUMMARY OF THE INVENTION

In order to combat subcutaneous CVC, disclosed herein is an applicator that can be advanced over an in-situ guidewire and into the subcutaneous tissue or tract prior to CVC placement or replacement, allowing sterilization of the subcutaneous tissue or tract before CVC introduction. The unique shape of the applicator allows sterilization of the skin surface as well as the subcutaneous tissue or tract of the original CVC that lies beneath the skin, which are difficult to reach and effectively sterilize with other applicators.

### BRIEF DESCRIPTION OF THE DRAWINGS

FIG. 1 is a top view of the applicator.

FIG. 2 is a perspective view of the applicator.

FIG. 3 is a side view of the applicator.

FIG. 4 is a front view of the applicator.

FIG. 4A is a cut-away view of the applicator.

FIG. 5 shows a perspective view of the combined dilator/applicator.

FIG. 6 shows a side view of the combined dilator/applicator.

FIG. 7 shows a front view of the combined dilator/applicator.

FIG. 7A shows a cut-away view of the combined dilator/applicator.

FIG. 8 shows a top view of a combined dilator/applicator with a medicament delivery device.

FIG. 9 shows a side view of the dilator/applicator with the medicament delivery device.

FIG. 10 shows a side view of the dilator/applicator with the medicament delivery device.

FIG. 11 shows an alternate side view of the dilator/applicator with the medicament delivery device.

FIG. 12 shows a perspective view of the dilator/applicator with the medicament delivery device.

### DETAILED DESCRIPTION OF THE INVENTION

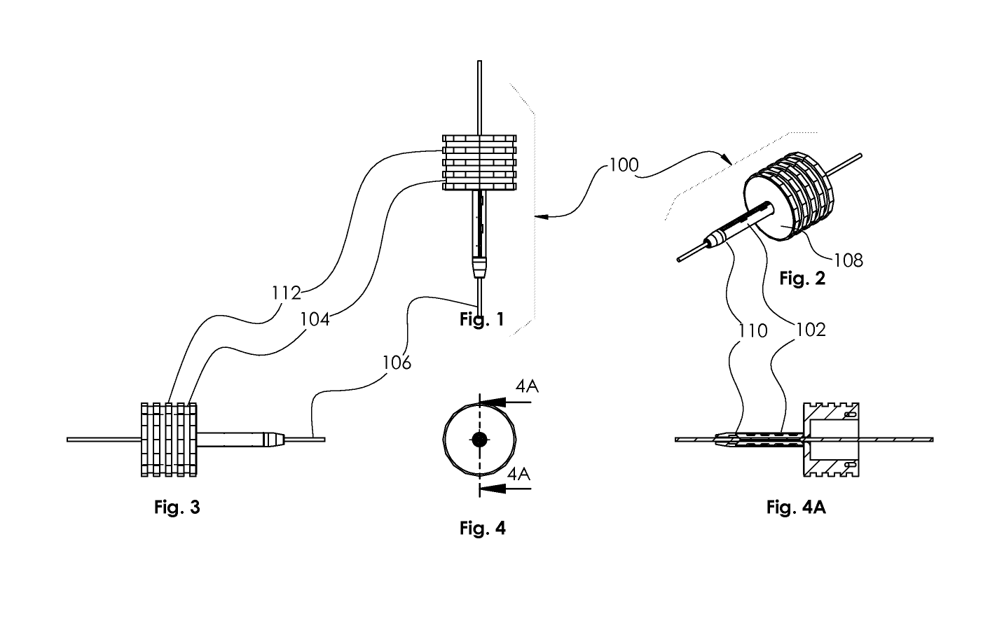

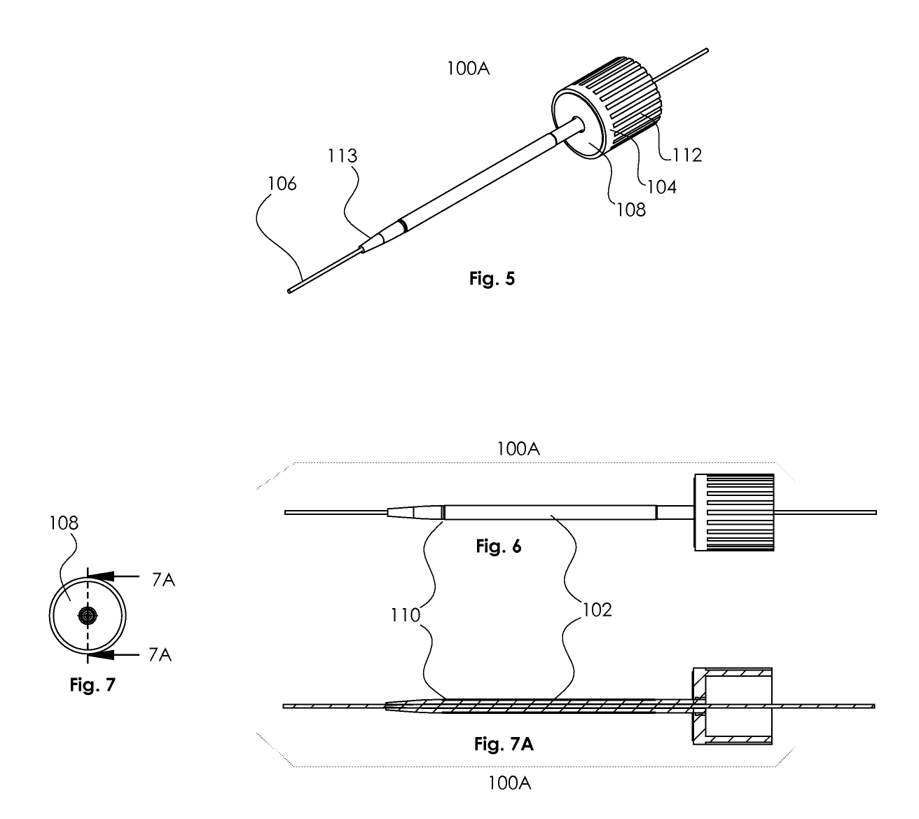

With reference to FIGS. 1-4A, shown is applicator 100, and FIGS. 5-7A shown is combined applicator/dilator 100A which comprises shaft 102 and thumbwheel 104. Shaft 102 is formed as a cylindrical hollow tube which is sized to advance over a guidewire 106 and fit snugly in the subcutaneous tract. Similarly, thumbwheel 104 is formed as a larger cylinder having a central channel similar in size to that of shaft 102. The greater diameter of thumbwheel 104 prevents the entirety of applicator 100 from inadvertently entering the subcutaneous tract.

In some embodiments, shaft 102 may extend the entire length of applicator 100, through thumbwheel 104, and form the inner channel of thumbwheel 104. The inside of thumbwheel 104 may be hollow or solid depending upon the manufacturing method used (e.g., thermoplastic molding) or the desired use of applicator 100.

Another possible implementation of applying medication or disinfectant to the outside and/or inside of shaft 102 is to include a reservoir in applicator 100 with a mechanism allowing the release of the medication or disinfectant. This reservoir will likely, but not necessarily, be located within the thumbwheel 104.

An outer surface of shaft 102 is covered with a fabric like cover or other medium which is capable of holding or absorbing antiseptic or other medication. A grommet 110 located at an end of shaft 102 may be used to prevent the fabric like cover or other medium from sliding off of applicator 100 during application of the antiseptic or other medication. Preferably, shaft 102 and grommet 110 are tapered for easy passage of applicator 100 into the subcutaneous tract of the patient. The fabric like cover or other medium may be applied to the surface of shaft 102 using any known methods such as adhesives, chemical bonding, etc.

In some embodiments, an inner surface of shaft 102 may also comprise a fabric like cover or other medium similar to that on the outer surface of shaft 102. This allows antiseptic or other medication to be applied to the inner surface of shaft 102 to treat/disinfect guidewire 106 as applicator 100 is advanced over guidewire 106.

A bottom surface 108 of thumbwheel 104 may also be covered with a fabric like cover or other medium for holding antiseptic or other medication. When applicator 100 is advanced to the skin surface, the bottom surface 108 is used to apply the antiseptic or medication to the skin surface as applicator 100 is rotated.

In one embodiment, a diameter of thumbwheel 104 is approximately 19 mm wide. The length of shaft 102 may be 16 mm with a diameter of 2.5 mm. These dimensions allow applicator 100 to be used on many different sized guidewires 106. However, it should be apparent that any dimensions for applicator 100 may be used depending on the desired application.

Preferably, applicator 100 is packaged in a single-use sterilized package similar to other medical equipment such as needles or syringes. Applicator 100 may come pre-soaked (i.e., with antiseptic/medication already applied) or the antiseptic/medication may be applied separately. For example, in certain situations, specialized medications or different combinations of medications may be desired.

In a preferred embodiment, the sidewalls of thumbwheel 104 have a textured surface 112 to allow for easy rotation of applicator 100 by use of a practitioner's thumb after applicator 100 has been advanced into the subcutaneous tract. The textured surface 112 also allows a practitioner to easily grip and advanced applicator 100 over the guidewire.

In order to use applicator 100 during CVC placement, the operating field (skin) is prepped with antimicrobial solution and sterilely draped. The practitioner accesses the target blood vessel with a needle through which a guidewire 106 is placed into the vessel of interest. The needle is removed, leaving guidewire 106 in-situ. A tissue dilator is then passed over the guidewire 106 to widen the subcutaneous tract. In certain embodiments, applicator 100A may serve as both an applicator and a tissue dilator simultaneously where tapered end 113 widens the opening as would a tissue dilator: and the tissue dilator step can be skipped. Applicator 100 is then passed over guidewire 106 (with shaft 102) first and into the subcutaneous tissue. As applicator 100 is advanced, any antiseptic/medication located on the inside of shaft 102 is passed to the surface of guidewire 106 for sterilization. The practitioner rotates applicator 100 using thumbwheel 104 to apply the antiseptic/medication to the surface of the skin of the patient from bottom surface 108 and the subcutaneous tract from the outer surface of shaft 102. Applicator 100 is then removed from guidewire 106 and the CVC is placed using guidewire 106. Guidewire 106 is removed leaving the new CVC in place. With the use of applicator 100, both the subcutaneous tissue and the guidewire are sterilized prior to CVC introduction, thereby reducing the risk of CVC contamination.

In order to use applicator 100 during over the wire exchange, the CVC field is prepped with a skin antiseptic and sterilely draped. The practitioner passes guidewire 106 through the CVC and removes it, leaving guidewire 106 in-situ. Applicator 100 is then passed over guidewire 106 (with shaft 102 first) and into the subcutaneous tract. As applicator 100 is advanced, any antiseptic/medication located on the inside of shaft 102 is passed to the surface of guidewire 106 for sterilization. The practitioner rotates applicator 100 using thumbwheel 104 to apply the antiseptic/medication to the surface of the skin of the patient from bottom surface 108 and the subcutaneous tract from the outer surface of shaft 102. Applicator 100 is then removed from guidewire 106 and a new CVC is placed using guidewire 106. Guidewire 106 is removed, leaving the new CVC in place. With the use of applicator 100, patients can benefit from the low risk of mechanical complications associated with the over the wire technique, without an appreciable increase in risk of CLABSI.

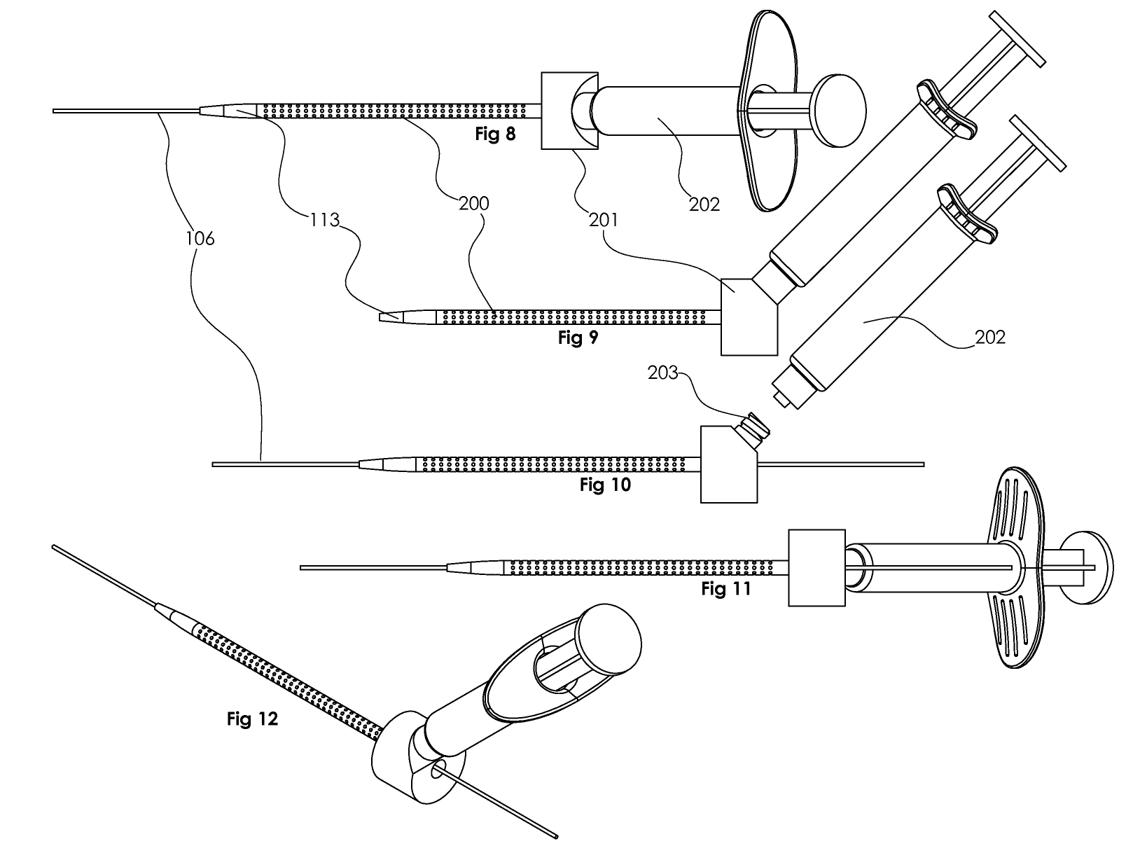

An alternate embodiment of applicator 100 is depicted in FIGS. 8-12. As shown, applicator 100 is fashioned of hard plastic with a central lumen 200 to allow applicator 100 to pass over guidewire 106. Alternative to perforations, central lumen 200 can also be manufactured from a porous material. The multiple perforations in central lumen 200 allow spread of injected antiseptic or medicament to reach subcutaneous tissues as well as guidewire 106.

In order to receive the medicament, applicator 100 comprises valve body 201 which has a port 203 allowing connection for a syringe 202 or other medicament delivery system. Port 203 may have a threaded connection for connecting to syringe 202. Also, port 203 may be provided with a cover when syringe 202 is not attached. Syringe 202 is used to inject medicament which is then dispersed through central lumen 200, coating its inner and outer surface. This allows for administration of medicament to both guidewire 600 and the subcutaneous tissues.

We claim:

1. An applicator comprising:
a hollow shaft having an inner surface, an outer surface, a proximal end, and a distal end;
a thumbwheel extending from the proximal end of the hollow shaft; and
an applicator surface located on the outer surface of the hollow shaft,
wherein the applicator surface is adapted to receive a sterilizing agent or a medication.

2. The applicator according to claim 1, further comprising:
a skin applicator surface on a bottom surface of the thumbwheel,
wherein the skin applicator surface is adapted to receive a sterilizing agent or a medication.

3. The applicator according to claim 1, wherein an outer surface of the thumbwheel comprises a textured surface.

4. The applicator according to claim 1, wherein the distal end of the hollow shaft is tapered.

5. The applicator according to claim 1, further comprising:
a grommet at a first end of the applicator surface located on the outer surface of the hollow shaft,
wherein the applicator surface extends from a bottom surface of the thumbwheel to the grommet.

6. The applicator according to claim 1, wherein a diameter of the thumbwheel is greater than a diameter of the hollow shaft.

7. The applicator according to claim 1, further comprising:
a guidewire applicator surface located on the inner surface of the hollow shaft, wherein the guidewire applicator surface is adapted to receive a sterilizing agent or a medication for treating a guidewire.

8. The applicator according to claim 1, wherein the hollow shaft and the thumbwheel are cylindrical.

9. The applicator according to claim 1, wherein an inner diameter of the hollow shaft is sized to advance the applicator over a guidewire to a skin surface.

10. The applicator according to claim 1, further comprising:
a central channel in a center of the thumbwheel,
wherein the central channel abuts the proximal end of the hollow shaft and extends an entire length of the thumbwheel.

11. The applicator according to claim 1, further comprising:
a dilator extending from the distal end of the hollow shaft,
wherein an outer surface of the dilator tapers over an entire length of the dilator.

12. The applicator according to claim 11, wherein an outer surface of the dilator comprises a dilator applicator surface, and
wherein the dilator applicator surface is adapted to receive a sterilizing agent or a medication.

13. The applicator according to claim 1, wherein the applicator surface is a fabric-like surface.

14. A method for performing sterilization using an applicator comprising:
advancing the applicator over a guidewire until a hollow shaft of the applicator enters into subcutaneous tissue of a subject;
rotating the applicator using a thumbwheel of the applicator that is external to the subject,
wherein the rotation passes a medication or a sterilizing agent from an external surface of the hollow shaft to the subcutaneous tissue; and
removing the applicator from the guidewire.

15. The method according to claim 14, wherein an inner shaft of the applicator is coated with a medication or a sterilizing agent which is passed to the guidewire as the applicator is advanced.

16. The method according to claim 14, wherein a bottom surface of the thumbwheel is coated with a medication or a sterilizing agent which is passed to a skin surface of the subject as the thumbwheel is rotated.

17. The method according to claim 14, further comprising:
placing a central venous catheter using the guidewire after the applicator is removed from the guidewire; and
removing the guidewire and leaving the central venous catheter.

18. An applicator comprising:
a hollow shaft having an inner surface, an outer surface, a proximal end, and a distal end, wherein the hollow shaft comprises a plurality of perforations;
a thumbwheel extending from the proximal end of the hollow shaft;
an applicator surface located on the inner surface and outer surface of the hollow shaft; and
a port in fluid connection with the hollow shaft for accepting a medicament, wherein injection of the medicament into the port by a delivery device causes delivery of the medicament to the applicator surface located on the outer surface of the hollow shaft through the plurality of perforations.
